(19) United States
(12) Patent Application Publication
(10) Pub. No.: US 2008/0103571 A1
(43) Pub. Date: May 1, 2008

(54) MEDICAL LEAD DELIVERY DEVICE

(76) Inventors: Ryan Thomas Bauer, Brooklyn Park, MN (US); Pedro A. Meregotte, Vadnais Heights, MN (US); John L. Sommer, Coon Rapids, MN (US); John B. Horrigan, Beverly, MA (US); Patrick P. Senarith, Circle Pines, MN (US); H. Allan Steingisser, Melrose, MA (US)

Correspondence Address: MEDTRONIC, INC., 710 MEDTRONIC PARKWAY NE, MINNEAPOLIS, MN, 55432-9924, US

(21) Appl. No.: 11/555,004

(22) Filed: Oct. 31, 2006

(51) Int. Cl. A61N 1/05

(52) U.S. Cl. 607/116

(57) ABSTRACT

The medical lead delivery device combines features of a guidewire and a stylet in order to more easily and quickly deliver a lead to the left ventricle of a patient's heart. The medical lead delivery device includes an elongated body, a controller, a first and second spring, and a sleeve. The elongated body includes a proximal end and a distal end. The controller is disposed at the proximal end and provides enhanced control of the distal tip of the elongated body.

### TECHNICAL FIELD

The present invention relates to medical devices and, more particularly, to delivery of implantable medical device leads.

### BACKGROUND

Most commercially available cardiac pacing and defibrillation leads are placed by means of a stylet which is inserted into a central lumen through the lead, and is used to assist in pushing the lead through the vascular system and guiding it to a desired location. Leads may also be placed by a guidewire extending entirely through the lead and out its distal end. This basic approach has been adapted to cardiac pacing leads and cardioversion leads as well, as disclosed in U.S. Pat. No. 5,003,990 issued to Osypka, U.S. Pat. No. 5,755,765 issued to Hyde et al, U.S. Pat. No. 5,381,790 issued to Kenasaka and U.S. Pat. No. 5,304,218 issued to Alferness.

### BRIEF DESCRIPTION OF DRAWINGS

Aspects and features of the present invention will be appreciated as the same becomes better understood by reference to the following detailed description of the embodiments of the invention when considered in connection with the accompanying drawings, wherein:

FIG. 1 is a block diagram of an implantable medical device;

FIG. 2 is a block diagram of a delivery device in a medical lead;

FIG. 3 is a cross-sectional view of a delivery device of FIG. 2;

FIG. 4 is an enlarged view of a proximal joint of the delivery device depicted in FIG. 3;

FIG. 5 is an enlarged view of a distal joint of the delivery device depicted in FIG. 3; and

FIG. 6 is an enlarged view of a tip joint of the delivery device depicted in FIG. 3.

### DETAILED DESCRIPTION

The present invention is directed to a delivery device that assists in placement of a medical lead in the left heart of a patient. The delivery device is a hybrid of a guidewire and a stylet. The medical lead delivery device includes an elongated body, a controller, a first and second spring, and a sleeve. The elongated body includes a proximal end and a distal end. The controller is disposed at the proximal end and provides enhanced control of the distal tip of the elongated body. In particular, the delivery device can be advanced beyond the tip of the lead to provide a “rail” for the medical lead to track. The first and second springs are coupled to the distal end of the elongated body. A sleeve is coupled to the elongated body and to the first and second springs through first, second and third solder elements. The delivery device eases delivery of a medical lead to the left ventricle of the heart. Additionally, a lower manufacturing cost exists to produce the delivery device.

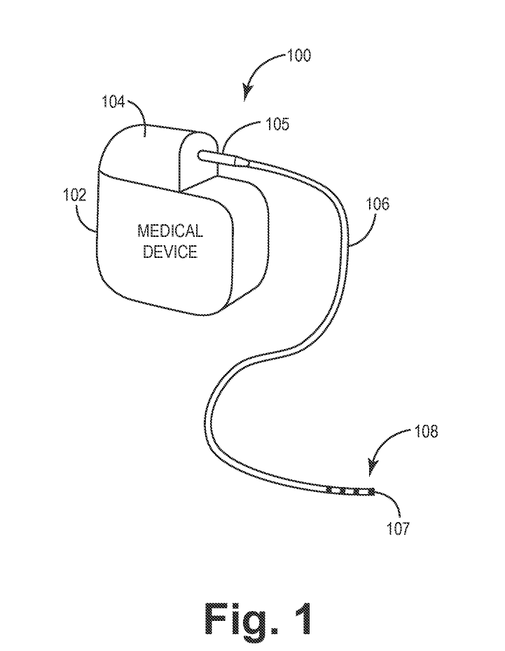

FIG. 1 depicts a medical device system 100. A medical device system 100 includes a medical device housing 102 having a connector module 104 that electrically couples various internal electrical components of medical device housing 102 to a proximal end 105 of a medical lead 106. A medical device system 100 may comprise any of a wide variety of medical devices that include one or more medical lead(s) 106 and circuitry coupled to the medical lead(s) 106. An exemplary medical device system 100 may take the form of an implantable cardiac pacemaker, an implantable cardioverter, an implantable defibrillator, an implantable cardiac pacemaker-cardioverter-defibrillator (PCD), a neurostimulator, or a muscle stimulator. Medical device system 100 may deliver, for example, pacing, cardioversion or defibrillation pulses to a patient via electrodes 108 disposed on distal end 107 of one or more lead(s) 106. In other words, lead 106 may position one or more electrodes 108 with respect to various tissue (e.g. cardiac tissue etc.) locations so that medical device system 100 can deliver pulses to the appropriate locations.

Lead 106 is provided with an elongated insulative lead body (e.g. insulative polymeric tube etc.), which carries a coiled conductor therein. Other lead body types may be substituted within the context of the present invention, including lead bodies employing multiple lumen tubes and/or stranded or braided conductors as disclosed in U.S. Pat. No. 5,584,873 issued to Shoberg et al, and incorporated herein by reference in its entirety. Alternatively, the lead may include additional conductors arranged either within a multi-lumen lead body or concentrically, as disclosed in U.S. Pat. No. 4,355,646 issued to Kallok et al and incorporated herein by reference in its entirety. Additional pacing electrodes, sensors, or defibrillation electrodes, may of course be added to the lead body and coupled to additional conductors.

At the proximal end of the lead body is a connector assembly (e.g. IS-1, IS-4 connector assemblies etc.) used in commercially available cardiac pacing leads. The connector assembly includes a conductive connector pin which is coupled by means of the conductor within the lead body to a tip electrode located at the distal tip of lead 106.

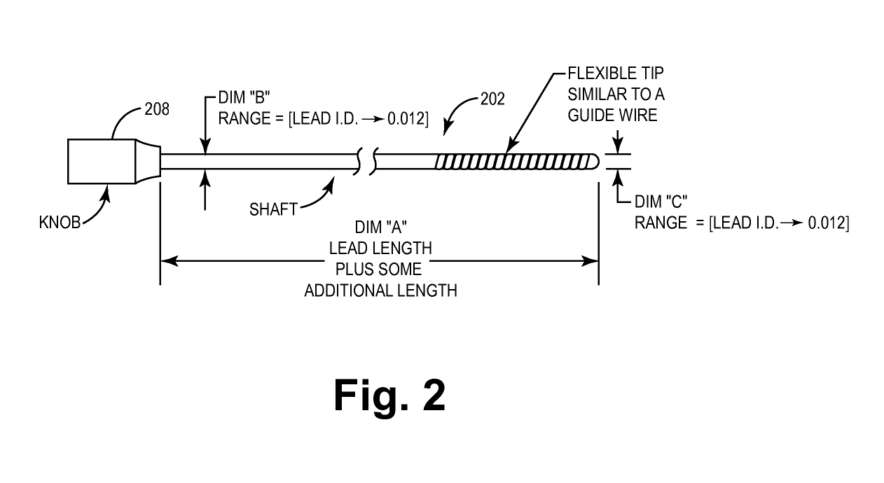

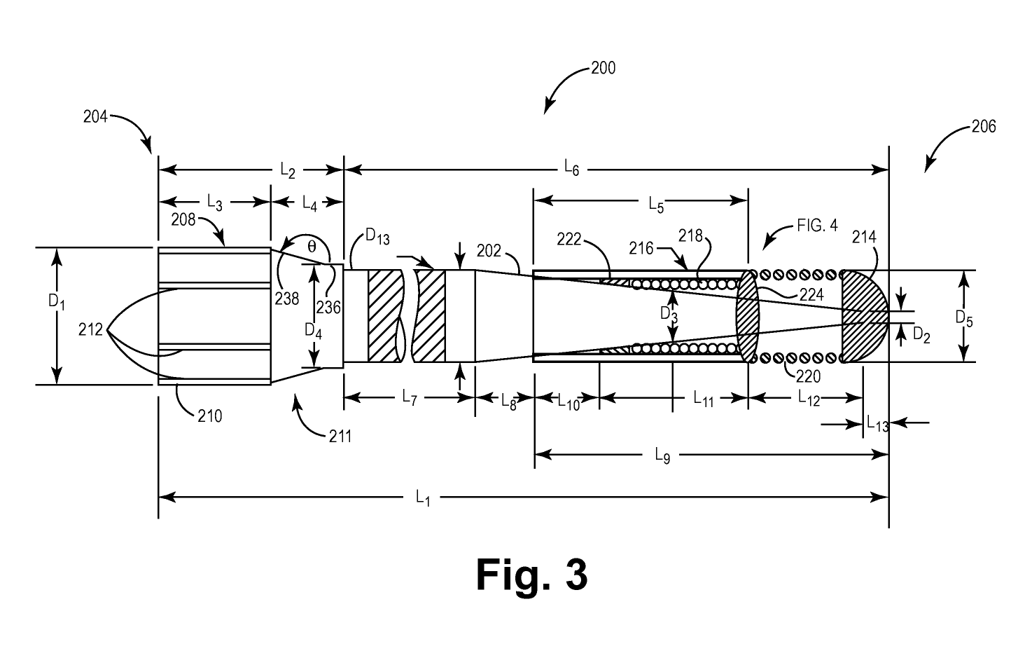

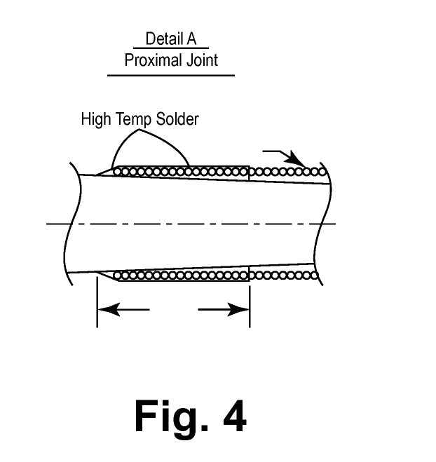

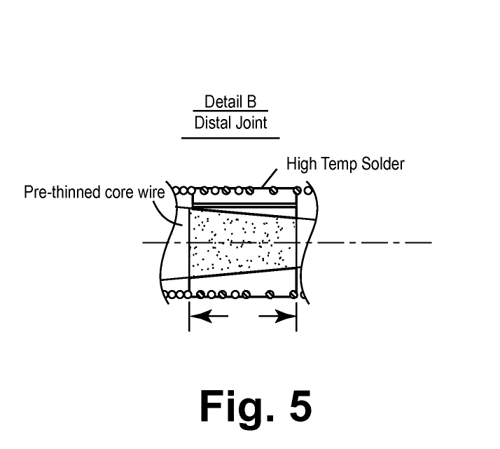

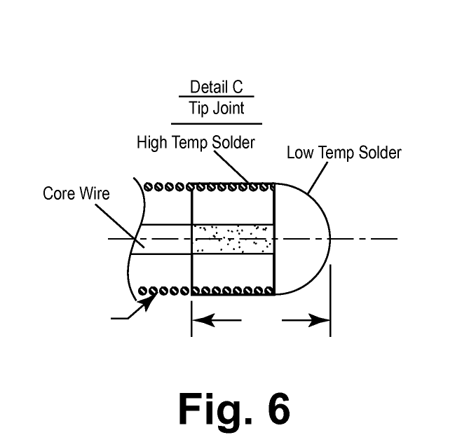

FIGS. 2-6 depict details of a delivery device 200 (or delivery wire) used to place lead 106 in a patient's body (e.g. left heart etc.). Delivery device 200 has a proximal end 204 and a distal end 206. Delivery device 200 comprises a controller 208, an elongated member 202, a sleeve 216, springs (or coils) 218, 220 and solder coupled to springs 218, 220 and to sleeve 216. Elongated member 202 comprises a conductive material (e.g. stainless steel, NiTiNOL (i.e. a family of Ni—Ti Alloys etc.)) with a length up to L1 and a diameter that ranges from D1 to D4. At proximal end 204 is controller 208. Controller 208 is an ergonomic knob configured to allow more control of the distal tip to elongated member 202 relative to lead 106. In particular, controller 10 assists in advancing delivery device 200 beyond the distal tip of lead 106 to provide a “rail” for the medical lead to track. In one embodiment, controller 208 is permanently attached to elongated member 202. In another embodiment, controller 208 is temporarily coupled to elongated member 202 to allow controller 208 to be removed from elongated member 202. For example, controller 208 may be screwed onto the proximal end 204 of elongated member 202.

In one embodiment, controller 208 comprises a gripping member 210 and a tapered distal end 211 with a length of about L2. Gripping member 210 is cylindrically shaped and includes a diameter that of about D1 and a length that extends L3. During insertion of a lead into a patient, gripping member 210 is held between the thumb and the forefinger of the person attempting to place the lead in the left heart. In one embodiment, gripping member 210 includes elongated recessed regions 212 to enhance the person's ability to hold gripping member 210. At the distal end of gripping member 210 is a tapered distal end 211. Tapered distal end 211 includes a diameter D4, a length L4, and angle θ formed by first and second sides 236, 238. Tapered distal end 211 of controller 208 is configured to receive the proximal end of elongated member 202. The proximal end of elongated member 202 includes a D13.

A distal portion of elongated member 202 is surrounded by cylindrical sleeve 216 with spring 218 disposed between an inner wall of sleeve 216 and elongated member 202. Sleeve 216 provides lubricity for moving within a lead body and coil alignment between springs 218, 220. Sleeve 216 extends a length of L5 and includes an inner diameter of Dₛₗₑₑᵥₑ. Solder 224 (also referred to as a second solder element) connects sleeve 216 to elongated member 202, and to springs 218, 220. Solder 224 is introduced over spring 218 and sleeve 216 at a high temperature.

Elongated member 202 extends a length of L6, which is comprised of regions defined by lengths L7, L8, and L9. The L7 region includes a diameter D13 whereas the L8 region is tapered at its distal end and contacts sleeve 216. The L8 region has a diameter that ranges from about D8ₛₘₐₗₗ to about D_{large}. The L9 region is tapered and includes regions L10, L11, L12, and L13. The L10 region includes a tapered section of elongated member 202 defined by a diameter that ranges from about D10ₛₘₐₗₗ to about D10_{large}. At the distal end of the L10 region is solder element 222. Solder element 222, also referred to as a third solder element, connects sleeve 216 with spring 218 and elongated member 202. Region L11 depicts spring 218 around elongated member 202. Region L11 includes a tapered section of elongated member 202 defined by a diameter that ranges from about D11ₛₘₐₗₗ to about D11_{large}. The L12 region extends from solder elements 224 and 214. The distal tip of elongated member 202 extends into solder 214 which increases isodymetry and body (or stiffness) to elongated member 200. Solder 214 has a diameter of D5 and is also referred to as the first solder element.

Springs 218, 220 are formed from any desired conductive material, selected based on the application of the elongated member being manufactured. Conductive material includes conductive metals or alloys, and/or conductive polymers. For example, springs 218, 220 may be formed from silver, platinum, gold, copper, a conductive alloy, or any other conductive material suitable for use in a medical lead.

Provided in Table 1 are the general dimensions for a delivery device 200 made to deliver 4 and 6 French leads.

TABLE 1
Dimensions of a delivery device.

| Element | Dimension of a delivery | Dimension of a delivery |
|---|---|---|
| designation | device for 4 French Lead | device for a 6 French Lead |
| L1 | 43.01 inches | 43.01 inches |
| L2 | 0.49 inches | 0.49 inches |
| L3 | | |
| L4 | | |
| L5 | 9.45 inches | 9.45 inches |
| L6 | 42.52 inches | 42.52 inches |
| L7 | | |
| L8 | | |
| L9 | | |
| L10 | | |
| L11 | | |
| L12 | 2.36 inches | 2.36 inches |
| L13 | | |
| D1 | 0.19 inches | 0.19 inches |
| D2 | .0024 inches | .0024 inches |
| D3 | .009 inches | .012 inches |
| D4 | | |
| D5 | .012 inches | .012 inches |
| D13 | .014 inches | .014 inches |

Another embodiment of length of L1 is about 34 inches. Another embodiment of length of L1 is about 51 inches. L1 can range from about 34 inches to about 51 inches.

Various embodiments of the invention have been described. These and other embodiments are within the scope of the following claims. Presented below are additional embodiments related to delivery device 200. For example, a slideable torque tool may be employed. This embodiment is implemented through the following: side loading occurs and/or a torque-limiting (or slip clutch mechanism)—engage with lead via a connector. In another embodiment, for ease of torquing delivery device 200, proximal end is configured with square (or on-rounded) cross-section or segmented round to non-round. In yet another embodiment, the delivery device is configured with alternating floppy and stiff areas. In still yet another embodiment, a coupling and decoupling via a lead and wire mechanism. In yet another embodiment, infusion wire with injection lumen and sideport—are able to inject contrast through the lumen. In yet another embodiment, a mechanism is employed for using a temperature sensitive alloy for lead fixation. In yet another embodiment, pacing wire may be unipolar and bi-polar configuration. This may include the following: a cathode range: 1.5 mm² to 15 mm²-5 mm² nominal and/or an anode range: 5 mm² to 30 mm²-10 mm² nominal. In yet another embodiment, a telescoping delivery device is employed. In yet another embodiment, delivery device includes a centering/loading tool.

1. A medical lead delivery device comprising:
an elongated body that includes a proximal end and a distal end;
a controller disposed at the proximal end;
a first and second spring coupled to the distal end of the elongated body; and
a sleeve coupled to the elongated body and to the first and second springs.

2. The medical lead delivery device of claim 1, wherein the controller configured to provide increased control over the distal tip of the elongated member.

3. The medical lead delivery device of claim 1, wherein the sleeve coupled to the elongated body and to the first and second springs through a first solder element.

4. The medical lead delivery device of claim 2, the sleeve coupled to the elongated body and to the first spring through a second solder element.

5. The medical lead delivery device of claim 3, the sleeve coupled to the elongated body and to the first spring through a third solder element.

6. The medical lead delivery device of claim 1, wherein the elongated body ranges in length from about 34 inches to about 51 inches.

7. The medical lead delivery device of claim 1, wherein the elongated body being about 34 inches.

8. The medical lead delivery device of claim 1 being a hybrid stylet and guidewire.

9. A medical lead delivery device comprising:
an elongated body that includes a proximal end and a distal end;
a controller disposed at the proximal end;
a first and second coil coupled to the distal end of the elongated body; and
a sleeve coupled to the elongated body and to the first and second coils,
wherein the controller configured to provide increased control over the distal tip of the elongated member.

10. The medical lead delivery device of claim 9, wherein the sleeve coupled to the elongated body and to the first and second coils through a first solder element.

11. The medical lead delivery device of claim 10, the sleeve coupled to the elongated body and to the first coil through a second solder element.

12. The medical lead delivery device of claim 10, the sleeve coupled to the elongated body and to the first coil through a third solder element.

13. The medical lead delivery device of claim 9, wherein the elongated body ranges in length from about 34 inches to about 51 inches.

14. The medical lead delivery device of claim 9, wherein the elongated body being about 34 inches.

15. The medical lead delivery device of claim 9 being a hybrid stylet and guidewire.
